(19) United States
(12) Patent Application Publication
(10) Pub. No.: US 2021/0130764 A1
(43) Pub. Date: May 6, 2021

(54) METHOD AND APPARATUS OF QR-CODED, PAPER-BASED, COLORIMETRIC DETECTION OF VOLATILE BYPRODUCT FOR RAPID BACTERIA IDENTIFICATION

(71) Applicant: THE TRUSTEES OF DARTMOUTH COLLEGE, Hanover, NH (US)

(72) Inventors: Alison BURKLUND, West Lebanon, NH (US); John X.J. ZHANG, Hanover, NH (US); Harrison SATURLEY-HALL, Hanover, NH (US)

(21) Appl. No.: 17/252,091

(22) PCT Filed: Jun. 11, 2019
(86) PCT No.: PCT/US2019/036502

§ 371 Date: Dec. 14, 2020

Related U.S. Application Data
(60) Provisional application No. 62/684,162, filed on Jun. 12, 2018.

(51) Int. Cl. C12M 1/34; G01N 31/22; G01N 33/52; G01N 21/78

(52) U.S. Cl. CPC C12M 41/34; G01N 31/22; G01N 2333/245; G01N 21/783; G01N 33/523

(57) ABSTRACT

A test device for indole concentrations in headspace gasses of bacterial cultures has a porous substrate imprinted with a wax barrier surrounding a test spot impregnated with p-dimethylaminocinnamaldehyde (DMACA). In embodiments, the test spot lies within a printed bar code and is configured to alter a reading of the bar code when the test spot darkens. The test device may optionally include a second test spot impregnated with Bromthymol Blue, Cobinamide, p-dimethylaminobenzaldehyde, or Chromotropic acid. The device is used by inoculating a sample into a culture; incubating the culture; inserting the test device into airspace of the culture, and observing the test spot for a color change indicative of indole presence.

### CROSS-REFERENCE TO RELATED APPLICATIONS

This application claims the benefit of priority to U.S. Provisional Application No. 62/684,162, filed Jun. 12, 2018, the entire content of which is incorporated herein by reference.

### FIELD

The present document relates to colorimetric detectors for volatile organic compounds produced by microorganisms in culture, and applies those detectors to detection and species identification of microorganisms. These detectors are of use in human and veterinary medicine, public health, sanitary inspection of food processing, water treatment, and sewage treatment, and other fields where rapid detection and identification of bacteria is desirable.

### BACKGROUND

There is a need for effective, inexpensive, rapid diagnostic tests for infections and infectious diseases, especially in point-of-care and low-resource settings.

Certain bacteria produce species-specific metabolic chemical byproducts. For example, Escherichia coli (E. coli) is known to produce certain indole compounds. Certain other microorganisms are known to produce other volatile compounds by fermentation during growth such as lactic acid, acetic acid, and ethyl alcohol.

E. coli is one of many bacteria that frequent the intestinal lumens of people and other mammals, including cattle. While many strains of E. coli seem innocuous, other strains of E. Coli, such as but not limited to E. coli O157H7, can cause serious hemorrhagic diarrhea and/or kidney failure. E. coli has also been implicated in approximately 16% of medically-significant sepsis cases, a significant percentage.

E. coli, as a coliform bacterium, is also often used as a marker of inadequate sewage treatment and disposal, and as a marker bacterium for contamination of food and water.

For these and other reasons there are many occasions where E. coli and other coliform bacteria must be rapidly grown and identified from potentially contaminated food, water, feces, urine, and blood.

A QR Code (quick response code) is a two-dimensional bar code having registration marks in its corners and bearing a message encoded with Reed-Solomon error-correcting codes. These codes are configured in multiple error-correcting code blocks interleaved and distributed within the two-dimensional bar code.

### SUMMARY

For rapid identification of Escherichia coli (E. coli) in bloodstream infections and cultures from other sources, we use a paper-based sensing platform which contains an array of well-defined printed detection areas each including colorimetric reagent p-dimethylaminocinnamaldehyde (DMACA) for the detection of volatile indole, a useful biomarker for E. coli identification. Our assay was able to quantitatively detect indole in the headspace (gas phase above the sample) of E. coli culture after twelve hours of growth (27.0+/−3.1 ppm), aiding in species-level identification earlier than some alternative methods.

To validate this paper-based assay, results were compared with headspace solid-phase microextraction (HS-SPME), two-dimensional gas chromatography, and time-of-flight mass spectrometry (GC×GC-TOFMS), which estimated indole concentration in E. coli culture to average 32.3+/−5.2 ppm after twelve hours of growth.

In a particular embodiment, the printed detection areas are distributed as pixels within a two-dimensional bar code configured to be readable with a two-dimensional bar code reader. In a particular embodiment, the two-dimensional bar code is a QR-code that directs to a “negative” site if no DMACA detection areas have changed color, and to a “positive” site if DMACA detection areas have changed color due to reaction with indole.

In an embodiment, a test device for indole concentrations in headspace gasses of bacterial cultures has a porous substrate imprinted with a wax barrier surrounding a test spot impregnated with p-dimethylaminocinnamaldehyde (DMACA). In embodiments, the test spot lies within a printed bar code and is configured to alter a reading of the bar code when the test spot darkens.

In an embodiment, the device is used by inoculating a sample into a culture; incubating the culture; inserting the test device into headspace of the culture, and observing the test spot for a color change indicative of indole presence.

### BRIEF DESCRIPTION OF THE FIGURES

FIG. 1A is a schematic illustration of an embodiment at system level.

FIG. 1B is a flowchart of forming and using a test device.

FIG. 1C is another schematic illustration of an embodiment at system level.

FIG. 2A is a sequence of cross-sectional diagrams showing fabrication of the test device.

FIG. 2B illustrates unused, negative, and positive resulting QR codes.

FIG. 2C illustrates exposing the sensor device to headspace gasses of a liquid culture.

FIG. 2D illustrates sensor spots in a 2-dimensional bar code with ones and zeroes adjacent.

FIG. 3A illustrates sensors exposed to varying concentrations of volatile indole and the associated color intensity output.

FIG. 3B illustrates color intensity as a function of indole concentration results

FIG. 3C illustrates relative inter-device reliability of color intensity output as a function of indole concentration results

FIGS. 4A and 4B illustrate results from three samples of E. coli in culture after 3, 6, 9, and 12 of culture.

FIG. 5 illustrates chromatogram area and extrapolated dissolved indole concentration of E. coli strains from HS-SPME, GC×GC-TOFMS analysis.

### DETAILED DESCRIPTION OF THE EMBODIMENTS

Paper is an attractive alternative to pressure-driven microfluidic platforms due to broad availability, ease of fabrication, low-cost, and its inherent ability to autonomously drive fluid flow with its embedded capillary pores. Sensing regions on the paper substrate can be defined through printing, which can also form patterns, such as bar codes, for digital readout. We have designed a paper microfluidic device that can rapidly detect indole production in Escherichia coli (E. coli) using a system that bypasses some traditional sample preparation steps.

Numerous colorimetric assays are employed in clinical microbiology laboratories to identify disease-causing organisms although some of these assays are time intensive due to sample preparation requirements. To minimize sample preparation, we target volatile indole, a degradation production of tryptophan, which is produced by E. coli, and is detectable in 98% of E. coli isolates. A sensitive colorimetric reagent used to detect indole is 3-[4-(dimethylamino) phenyl] prop-2-enal, known as p-dimethylaminocinnamaldehyde (DMACA). DMACA reacts with indole to form a blue-green substance, while unreacted DMACA is white. Current DMACA assays require that E. coli first be isolated from the biological sample, generally requiring an overnight culture step. Following this, an isolated colony is physically smeared onto filter paper saturated with the reagent. The DMACA reacts with any indole in the colony and turns a bluish green.

In this work, we have taken DMACA reagent and adapted its use to detect indole in gas-phase, effectivity eliminating time-intensive colony-isolation and smearing steps.

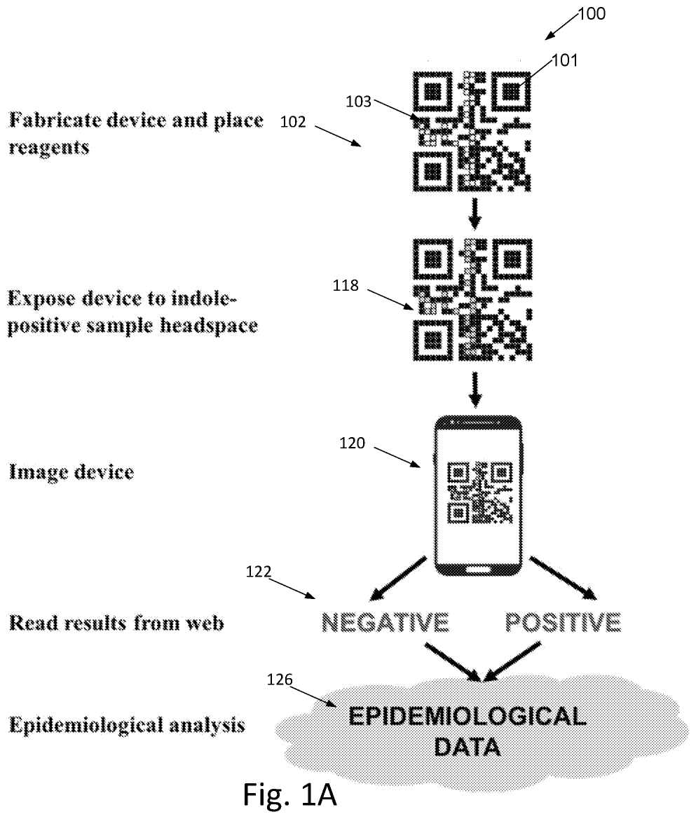

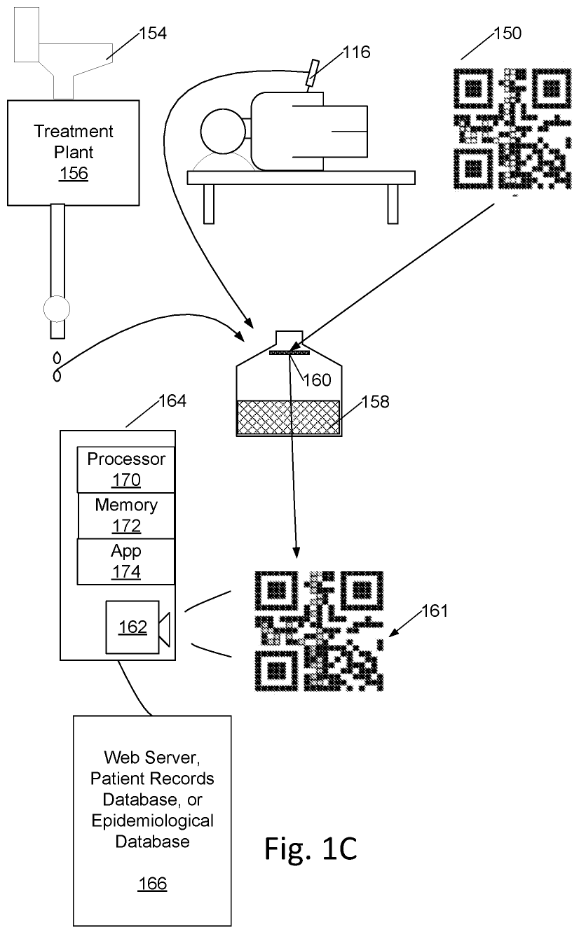

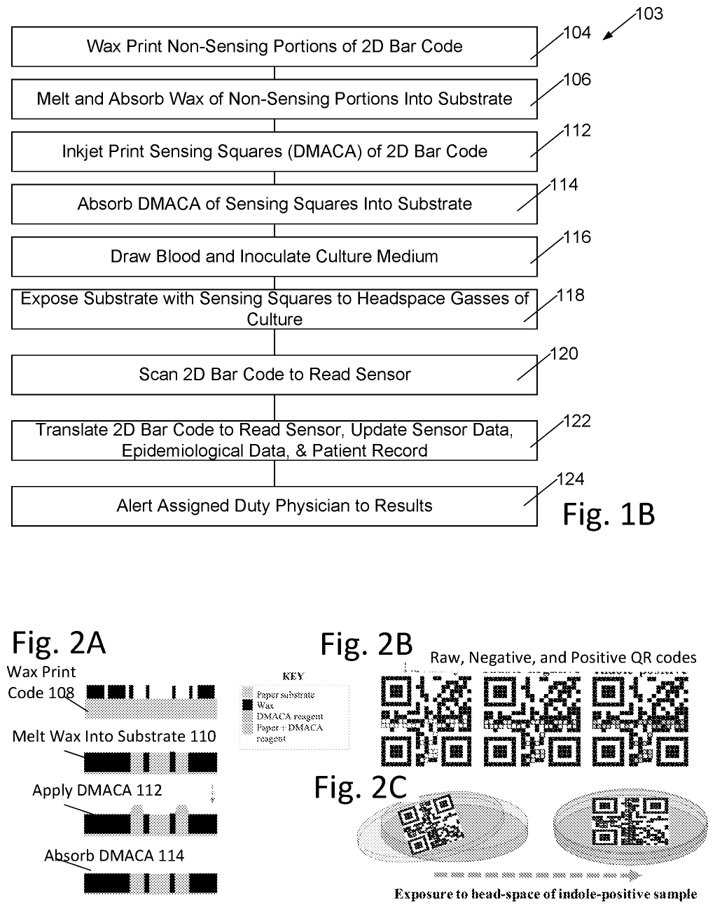

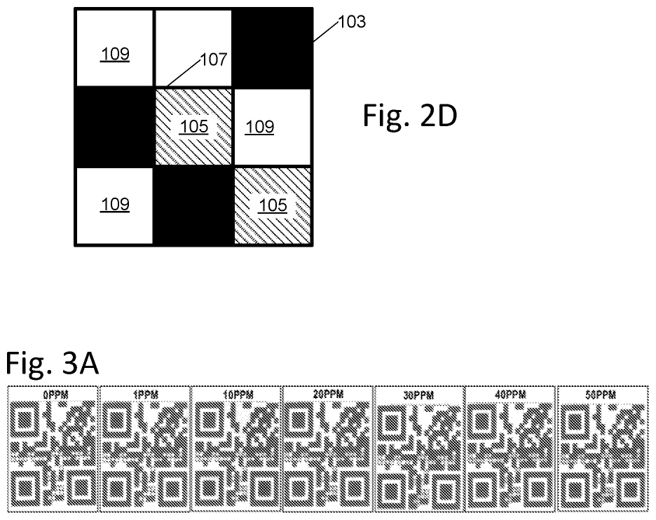

With reference to FIGS. 1A, 1B, 1C and 2A, a method 100 of testing for presence of E. coli in blood, water, tertiary treated sewage, or swabbed material from an agricultural facility such as a vegetable processing plant or slaughterhouse, begins by forming 102 a sensing device 150 and placing reagents thereon. Forming the sensing device is done by using a inkjet wax printer (Xerox ColorQube 8580) to print 104 non-sensing portions of an image incorporating registration marks 101, in an embodiment the image incorporating registration marks is a 2-dimensional bar code including multiple black code squares 103 on a white background. In a particular embodiment, the 2-dimensional bar code is a QR code incorporating at least two strings of characters grouped into two separate error-correcting code groups, a first group that incorporates a test identification and serial number, and a second group that incorporates a web address of a negative test report. The non-sensing portions of the image, including black QR code squares with the black wax registration marks, is printed using a inkjet wax printer onto a porous substrate that, in a particular embodiment, is formed of a Whatman #1 white chromatography paper. In a particular embodiment, black wax is also printed with a thermal wax printer to define borders of reagent test spots. In this embodiment, the black wax forms, when heated and absorbed into the substrate, a wax barrier defining size and shape of test spots to be impregnated with DMACA.

A stock solution is prepared from 10 grams of DMACA in 100 ml of 37% hydrochloric acid and 900 ml of water. Reagent test spots begin as unprinted bare-paper squares 105 within the image outlined with black printed wax lines 107 or adjacent black wax squares, see FIG. 2D. Only certain preselected unprinted, bare-paper, squares become reagent test spots, additional unprinted spots 109 are constant-zero bits of the 2-dimensional bar code.

The wax-printed image and registration marks are then heated 106 to ensure absorption of the wax into the porous substrate. Initially the wax lies atop 108 the substrate, but when melted by heating to 120 C for two minutes is absorbed 110 into the substrate. The black wax squares and black-printed wax lines 107 form barrier walls around reagent test spots 105.

Next, at least one unprinted bare-paper square 105 within the 2-dimensional bar code is printed 112, using an inkjet printer, with approximately 5 microliters of the DMACA reagent stock solution per 2 millimeter square spot. In an embodiment, multiple bare-paper squares 105 within the bar code are printed in a predetermined pattern and become sensing squares. The predetermined pattern of sensing squares is chosen such that, if all reagent-printed squares 105 became black, when the 2-dimensional bar code is read and the second code group is corrected, the second code group decodes as a second, positive, web address.

The DMACA reagent is permitted to absorb 114 into the paper substrate, but remains confined by the wax barrier walls to reagent test spots 105, the paper substrate with black wax printing and DMACA-impregnated spots becomes a formed sensor.

The formed sensors are dried for use. In an alternative embodiment, the DMACA reagent is stored in bubble that is popped by a lab technician or other user to apply fresh DMACA reagent to the chromatography or filter paper shortly before use.

In an embodiment of a method for detecting pathogens in treated sewage, sewage from sanitary facilities 154, as processed by a treatment plant 156, is sampled and grown in culture 158 on tryptic soy broth (TSB) media from Becton Dickinson.

In an embodiment of a method 103 of aiding in the diagnoses of bloodstream infections, a blood sample is drawn 116, and grown in culture 158 by inoculating it into a blood-culture medium enriched in tryptophan, and incubated. In an alternative embodiment (not shown), a urine sample is collected and inoculated into a urine-culture medium enriched in tryptophan and incubated. In an alternative embodiment (not shown), a swabbed sample of material from an agricultural facility is inoculated into a culture medium enriched in tryptophan and incubated. In an alternative embodiment, a sample of treated sewage is taken and inoculated into a culture medium enriched in tryptophan and incubated. In all four embodiments, the incubation period is about 12 hours to provide sufficient growth to affirm the presence of one or more bacteria in the blood, urine, swabbed sample, or sewage sample. The median time to blood culture positivity in patients with E. coli bacteremia is approximately twelve hours. Each culture has a test device 150, 103, 160 placed in airspace above the culture media. In embodiments, a second QR code attached to a lid or to a sidewall of a tube bearing a sample identification and may bear a patient identification associated with the culture.

The formed sensor is exposed to headspace gas of the incubated culture. In an embodiment, the formed sensor 150, 103, 160 is positioned within the headspace of the incubated culture for the entire incubation time. During exposure to the headspace gas, the reagent test spots 105 change color from near-white to a darker color in presence of indole gasses, and remain near-white if no indole gasses are present.

Next, the formed and exposed sensor 161 is read. In some embodiments, the sensor is visually read by medical personnel, in other embodiments the bar code including the sensing spots is imaged or scanned 120, as by a camera 162 of a cell phone 164 or bar code scanning device, and the second code group of the QR code being decoded and used as a web address to access a web page. A cell phone 164 used for scanning the QR code or other bar code includes a processor 170 with memory 172, the memory containing a QR code or other bar code reading application 174. If no indole gasses were present, the web page read is a negative-result web page; if indole gasses were present the dark color of reagent test spots transforms the web page referenced by the code group and read 122 to the scanning device into a positive-result web page address. In embodiments where a bar code of other types is used, the bar-code scanning device reads and interprets the code.

In the blood culture method, the second QR code bearing sample identification is then read, this second QR code incorporates a patient identification and the patient record 166 for that patient is updated with the positive or negative test result on a server; if a positive result is found additional actions may be taken, for example an on-call physician may be paged 124 with the rest result and an epidemiological record may be updated 126

Following blood culture positivity, a variety of molecular and traditional culture-based methods are used to identify the sepsis-causing organism(s), which can take anywhere between 2 to 72 additional hours. With additional organism identification information at an earlier time point close to the time of culture positivity, clinicians may more rapidly narrow antibiotic therapy to more likely effective and lower cost medications, thereby lowering health care costs and decreasing patient mortality rates.

Diagnosis of bloodstream infections (sepsis) represents one application for the proposed assay. E. coli accounts for approximately 16% of all bloodstream infections in the United States. Although indole is produced by E. coli, it is not produced by most other highly prevalent sepsis-causing organisms.

In an alternative embodiment, a 1-dimensional bar code is used instead of a 2-dimensional bar code, the bar code being printed with black wax on white chromatography or filter paper, the wax driven into the paper with heat, and with DMACA deposited on and impregnated selectively into unprinted bars of the bar code; the DMACA pattern being such that a scan of the bar code can determine a positive (indole present) from a negative (indole absent) test result.

In an alternative embodiment having greater sensitivity or dynamic range, DMACA-impregnated spots on the paper substrate are read with a colorimetric reader, this technique may allow detection of indole at as little as 1 ppm.

In an embodiment, our paper microfluidic colorimetric assay detected indole in the headspace of three strains of E. coli each growing in liquid media. Our results suggest that this assay can be quantitative, given an observed linear relationship between indole concentration and mean gray value intensity of the assay regions. Further, our assay detected indole in E. coli culture after twelve hours of growth. As a reference method, headspace solid-phase microextraction (HS-SPME), and two-dimensional gas chromatography time-of-flight mass spectrometry (GC×GC×TOFMS) was carried out on paired samples to allow both quantitative analysis and evaluation of device performance. The proposed system holistically integrates an inexpensive platform, and a methodological redesign of an existing colorimetric assay.

In order to assess the limit of detection and clinical relevance of this platform, experiments were performed using indole standard solutions along with three indole-producing E. coli strains. Pseudomonas aeruginosa (a non-indole producing bacterial species) was employed as a negative control.

Assay Exposure to Indole Standard Solution.

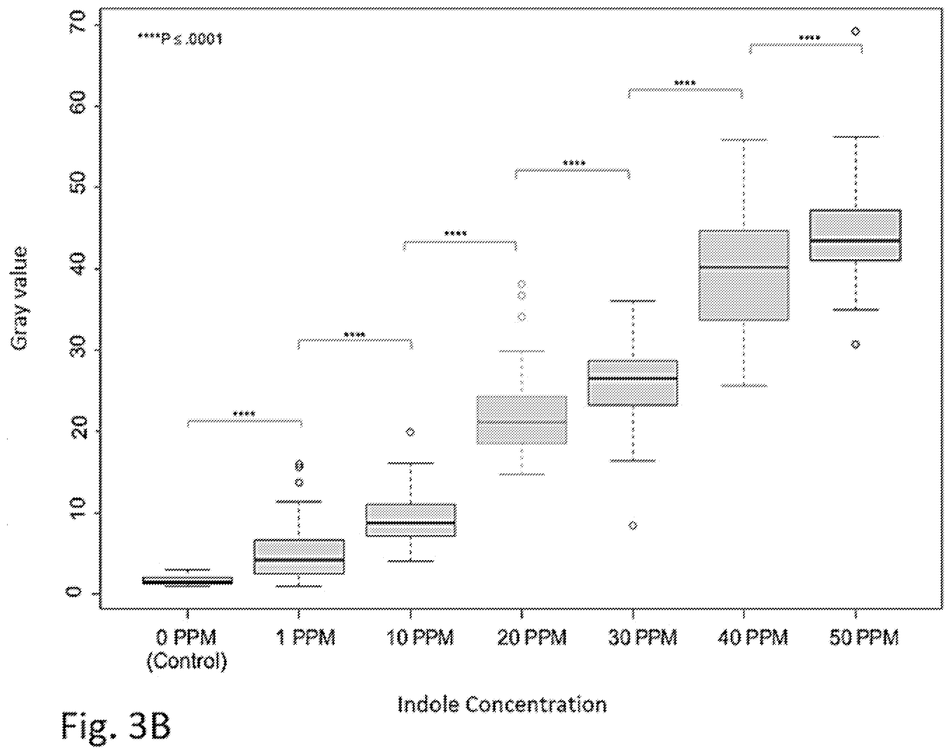

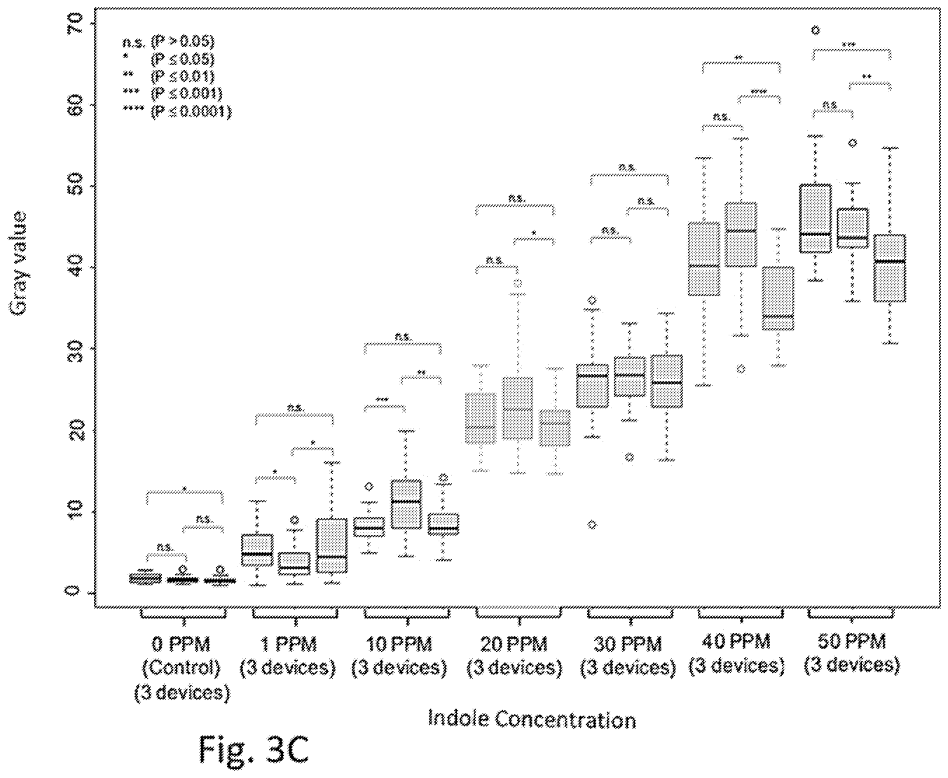

In order to assess dynamic range of the assay, devices were exposed to the headspace of indole analytical standard solutions with known concentrations, ranging from 1 to 50 ppm. Prior to any data analysis and image processing, the first evident color change was observed at 10 ppm (FIG. 3A). By employing some basic image processing tools, the mean gray value intensity for each assay region on the device was calculated. Since each device contains 30 unique assay regions, 30 data points were obtained per device. By averaging these 30 intensity values, the mean color intensity of the device when exposed to a 1 ppm standard solution was found to be significantly greater than color intensity observed at 0 ppm (FIG. 3B). Depending on the specific application of this assay, this increased sensitivity could be of value and is achievable with a colorimeter-read sensor including DMACA-impregnated spots on chromatography or filter paper. Additionally, the results from this analysis indicate that the mean color intensity correlates linearly with dissolved indole concentration. Next, in order to understand the effects of inter-device variability, three devices per concentration value were independently evaluated. The results of this analysis, which show that inter-device variability does not have a major effect on the mean gray value intensity, are shown in FIG. 3C. Importantly, these experiments also confirmed the feasibility of this novel assay methodology.

Assay Exposure to E. coli Culture Headspace

Next, the feasibility and clinical utility associated with employing this assay to detect indole production in E. coli in vitro was evaluated. Specifically, indole production was measured in three strains of E. coli:

1) E. coli K12 (clinical isolate),

2) E. coli ATCC 25922 (reference isolate), and

3) E. coli ATCC 43890 (reference isolate).

Following an overnight pre-culture step, between 27 and 55 CFU/mL were inoculated into sterile tryptic soy broth (TSB). This low inoculation dose was intentionally chosen to mimic the low cell concentration in patients with bloodstream infections. Further, TSB was intentionally selected as the culture media, as it is most similar to the proprietary media found in blood culture bottles. In order to diagnose sepsis, a blood culture is first required to affirm the presence of a pathogen in the bloodstream, but provides no species-level information. Given that the average time to blood culture positivity is approximately twelve hours, time points were selected to aid in bacterial species identification either prior to blood culture positivity (3 hours, 6 hours, 9 hours), or at worst, at the time of culture positivity (12 hours).

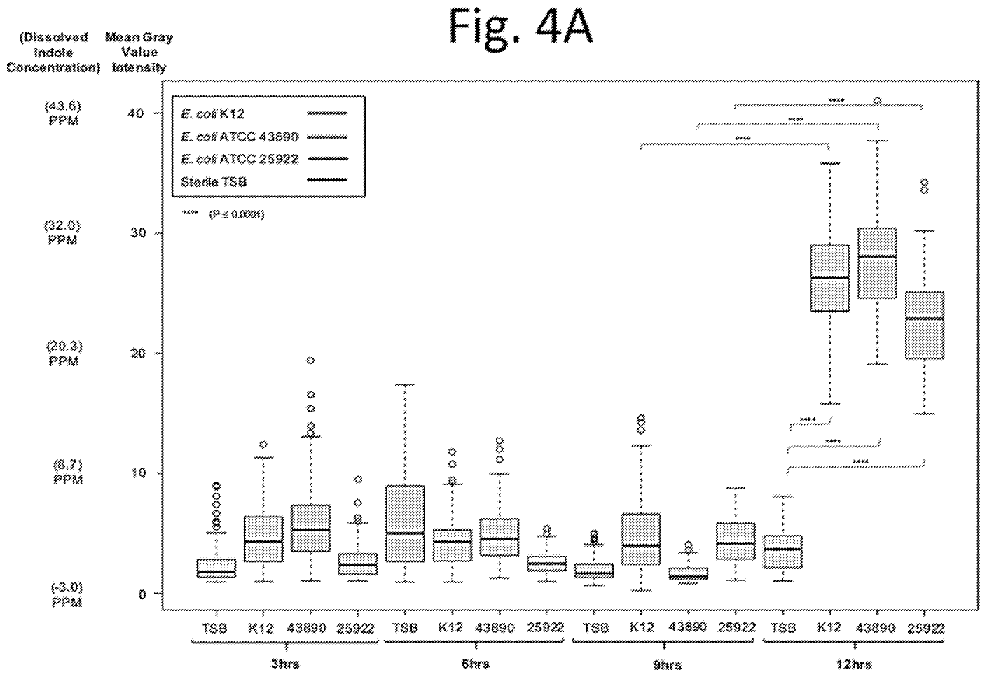

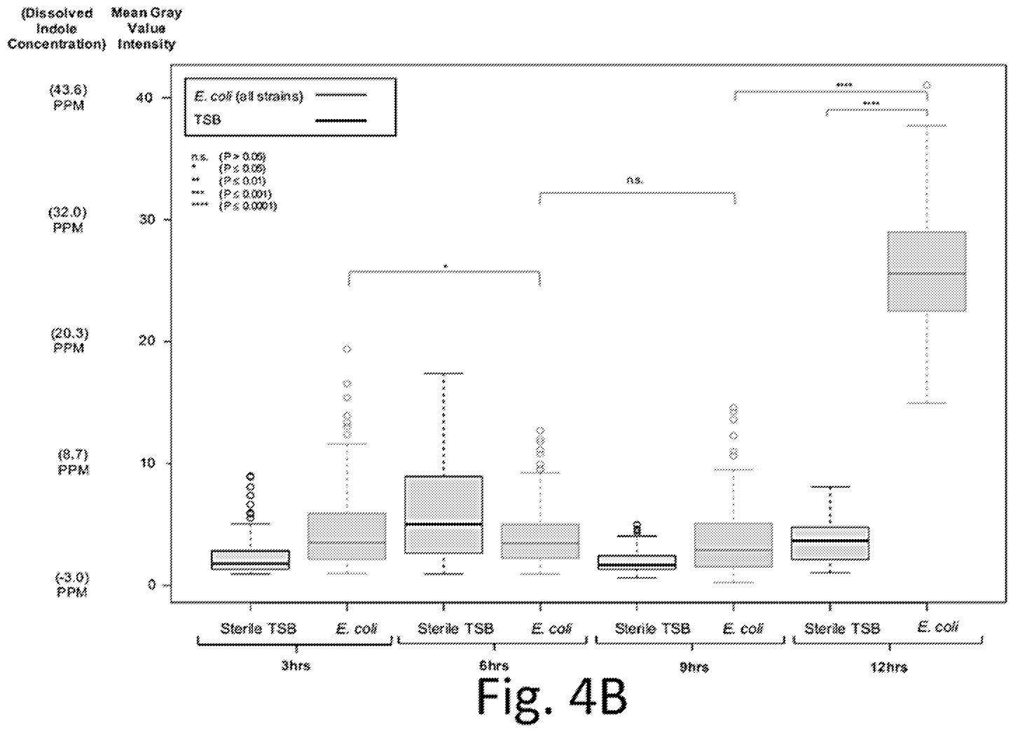

No visible change in color was observed in any of the devices exposed to the headspace of E. coli culture supernatant at 3 hours, 6 hours, and 9 hours. This was true for all three E. coli strains evaluated. That said, at 12 hours a significant shift in color intensity and pigmentation was observed. The results of these observations for each E. coli strain are shown in FIG. 4A. Remarkably, this suggests that indole production in an E. coli positive blood culture could be identified at or near the time of blood culture positivity. FIG. 4B illustrates observed mean gray value intensities for all 3 E. coli strains in combination. No color change was observed at any of the above specified time-points in the negative control, Pseudomonas aeruginosa (a non-indole producing bacterial species).

Quantification of Indole Production in E. coli with HS-SPME and GC×GX-TOFMS

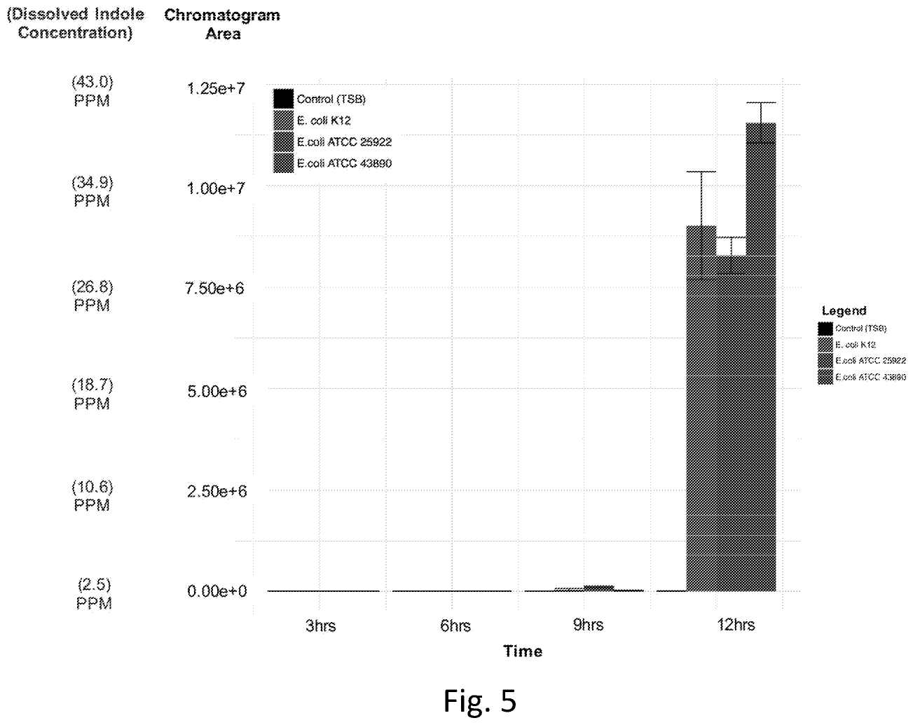

To further assess the performance of the assay, the indole concentration in E. coli culture supernatant was quantified using routine headspace solid-phase microextraction (HS-SPME) approach coupled to comprehensive two-dimensional gas chromatography time-of-flight mass spectrometry (GC×GC-TOFMS), as illustrated in FIG. 5. The dissolved indole concentrations measured using this high sensitivity, high cost system were remarkably similar to those obtained by the paper microfluidic assay, further suggesting that the paper assay is both quantitative and accurate. Due to the high sensitivity and cost associated with this analytical instrumentation, it is not surprising that indole was detected at an earlier than observed in the paper device. Although this could be a beneficial methodology to employ in state-of-the-art clinical microbiology facilities to enable indole detection, this type of instrumentation is not feasible to employ in lower income countries, or facilities that do not have the funding to support this type of instrumentation.

### Alternative Embodiments

It is expected that the paper-based colorimetric sensor for identification of bacteria grown in culture and herein described may be developed to identify additional bacterial species by use of substrates added to culture media and colorimetric reagents as described in table 1 below, 5 microliters of each of these reagents is applied to two-by-two millimeter test spots separate from the DMACA test spot:

TABLE 1

| Substrate | Bacterial | | |
|---|---|---|---|
| added to | species | | |
| culture | of Interest | Volatile gas | |
| medium | Detected | in headspace | Colorimetric reagent |
| Urea | Urease | Ammonia | Bromthymol Blue 0.01- |
| | producers: | | 0.1% by weight in water. |
| | Proteus spp., | | |
| | Helicobacter | | |
| | pylori | | |
| None | Pseudomonas | Hydrogen | Cobinamide 10-100 |
| | aeruginosa | Cyanide | micromolar in water. |
| None | Pseudomonas | 2-Amino- | 1% p-dimethylamino- |
| | aeruginosa | acetophenone | benzaldehyde |
| | | | in methanol |
| None | E. Coli | Methanol | Chromotropic acid, 1-10% |
| | | | by weight in water. |

In an embodiment, in addition to one or more DMACA-saturated test spots on the paper, there are additional test spots saturated with another colorimetric reagent listed in table 1; in yet another embodiment there are test spots bearing each of the colorimetric reagents listed in table 1. The paper is inserted into the headspace above the bacterial culture and incubated as previously described, and read optically to determine which, if any, test spots change color upon reacting with volatile gasses emitted by cultured bacteria into the airspace.

Conclusion

We discuss a novel paper microfluidic colorimetric assay, which is capable of colorimetric detection of indole in the gas phase on a paper substrate. This device is extremely low-cost, requires no sample preparation, can be fabricated using inkjet printing technology, and has potential for digital readout. We demonstrate this platform's functionality within the context of a specific and novel application: volatile indole detection to assist in the rapid identification of E. coli in bloodstream infections. To the best of our knowledge, no prior work has demonstrated the use of a paper substrate saturated with DMACA to detect indole from the headspace of a liquid culture. Not only did we demonstrate the ability to detect indole in the headspace of E. coli culture using a paper device, we also demonstrated that this assay can be quantitative, given the observed linear relationship between dissolved indole concentration and mean gray value intensity. This type of information could be useful in employing the assay for other diagnostic applications. For example, in the diagnosis of urinary tract infections (UTIs), bacterial load is a key variable affecting the diagnosis and treatment regimen.

Combinations

The test device features herein disclosed may be present in various combinations in different embodiments of the device and method. Combinations anticipated by the inventors include:

A test device designated A for indole concentrations in headspace gasses of bacterial cultures including a porous substrate imprinted with a wax barrier surrounding a test spot impregnated with p-dimethylaminocinnamaldehyde (DMACA).

A test device designated AA including the test device designated A wherein the test spot is a spot within a printed bar code configured to alter a reading of the bar code when the test spot darkens.

A test device designated AB including the test device designated AA The test device for indole concentration of claim 2 wherein the bar code is a two dimensional quick-response (QR) code configured to reference a negative-result internet page unless the test spot darkens, whereupon the bar code is a QR code configured to reference a positive-result internet page.

A test device designated AC including the test device designated A, AA, or AB wherein the test spot is formed by depositing about five microliters of 1% DMACA by weight in 3.7% hydrochloric acid, in a particular embodiment this test spot is two by two millimeters.

A test device designated AD including the test device designated A or AC The test device of claim 1 configured for colorimetric reading.

A test device designated AE including the test device designated A, AA, AB, AC, or AD further including a second test spot impregnated with a colorimetric reagent selected from the group consisting of Bromthymol Blue, Cobinamide, p-dimethylaminobenzaldehyde, and Chromotropic acid.

A method designated B of testing for E. Coli in a sample includes inoculating the sample into a culture; incubating the culture; inserting a test device comprising a porous substrate imprinted with a wax barrier surrounding a test spot impregnated with p-dimethylaminocinnamaldehyde (DMACA). into a headspace of the culture; and observing the test spot for a color change indicative of indole presence in a headspace of the culture.

A method designated BA including the method designated B wherein the observing the test spot for a color change is performed with a colorimeter.

A method designated BB including designated B wherein the test spot is formed as a portion of a bar code, the bar code configured to be altered by darkening of the test spot.

A method designated BC including the method designated B, BA, or BB wherein the test device further comprises a second test spot impregnated with a colorimetric reagent selected from the group consisting of Bromthymol Blue, Cobinamide, p-dimethylaminobenzaldehyde, and Chromotropic acid.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

1. A test device for indole concentrations in headspace gasses of bacterial cultures comprising:
a porous substrate imprinted with a wax barrier surrounding a test spot impregnated with p-dimethylaminocinnamaldehyde (DMACA).

2. The test device for indole concentration of claim 1 wherein the test spot is a spot within a printed bar code configured to alter a reading of the bar code when the test spot darkens.

3. The test device for indole concentration of claim 2 wherein the bar code is a two dimensional quick-response (QR) code configured to reference a negative-result internet page unless the test spot darkens, whereupon the bar code is a QR code configured to reference a positive-result internet page.

4. The test device of claim 1 wherein the test spot is formed by depositing about five microliters of 1% DMACA by weight dissolved in 3.7% hydrochloric acid.

5. The test device of claim 1 configured for colorimetric reading.

6. The test device of claim 1 further comprising a second test spot impregnated with a colorimetric reagent selected from the group consisting of Bromthymol Blue, Cobinamide, p-dimethylaminobenzaldehyde, and Chromotropic acid.

7. A method of testing for E. coli in a sample comprising:
inoculating the sample into a culture;
incubating the culture;
inserting a test device comprising a porous substrate imprinted with a wax barrier surrounding a test spot impregnated with p-dimethylaminocinnamaldehyde (DMACA). into a headspace of the culture; and
observing the test spot for a color change indicative of indole presence in a headspace of the culture.

8. The method of claim 7 wherein the observing the test spot for a color change is performed with a colorimeter.

9. The method of claim 7 wherein the test spot is formed as a portion of a bar code, the bar code altered by darkening of the test spot.

10. A method of testing for bacterial presence in a sample comprising the method of claim 7 wherein the test device further comprises a second test spot impregnated with a second colorimetric reagent selected from the group consisting of Bromthymol Blue, Cobinamide, p-dimethylaminobenzaldehyde, and Chromotropic acid.

11. The method of claim 10 wherein the second colorimetric reagent is Bromthymol Blue.

12. The method of claim 10 wherein the second colorimetric reagent is cobanimide.

13. The test device of claim 2 further comprising a second test spot impregnated with a colorimetric reagent selected from the group consisting of Bromthymol Blue, Cobinamide, p-dimethylaminobenzaldehyde, and Chromotropic acid.

14. The test device of claim 3 further comprising a second test spot impregnated with a colorimetric reagent selected from the group consisting of Bromthymol Blue, Cobinamide, p-dimethylaminobenzaldehyde, and Chromotropic acid.

15. The test device of claim 4 further comprising a second test spot impregnated with a colorimetric reagent selected from the group consisting of Bromthymol Blue, Cobinamide, p-dimethylaminobenzaldehyde, and Chromotropic acid.

16. The test device of claim 5 further comprising a second test spot impregnated with a colorimetric reagent selected from the group consisting of Bromthymol Blue, Cobinamide, p-dimethylaminobenzaldehyde, and Chromotropic acid.

17. A method of testing for bacterial presence in a sample comprising the method of claim 8 wherein the test device further comprises a second test spot impregnated with a second colorimetric reagent selected from the group consisting of Bromthymol Blue, Cobinamide, p-dimethylaminobenzaldehyde, and Chromotropic acid.

18. A method of testing for bacterial presence in a sample comprising the method of claim 9 wherein the test device further comprises a second test spot impregnated with a second colorimetric reagent selected from the group consisting of Bromthymol Blue, Cobinamide, p-dimethylaminobenzaldehyde, and Chromotropic acid.
